(19) United States
(12) Patent Application Publication
(10) Pub. No.: US 2025/0253039 A1
(43) Pub. Date: Aug. 7, 2025

(54) SYSTEM AND METHOD FOR MONITORING AND RECORDING SURGICAL PROCEDURES TO IMPROVE EFFICIENCY WITH REAL-TIME ALGORITHMIC ANALYSIS AND ASSISTIVE VIDEO OVERLAYS

(71) Applicant: Jack Wade, La Jolla, CA (US)

(72) Inventor: Jack Wade, La Jolla, CA (US)

(21) Appl. No.: 18/434,679

(22) Filed: Feb. 6, 2024

(51) Int. Cl. G16H 40/60; G16H 15/00; G16H 30/20

(52) U.S. Cl. CPC G16H 40/60; G16H 15/00; G16H 30/20

(57) ABSTRACT

A system for recording surgical environments and procedures with millisecond accuracy to ensure efficiency with real-time algorithmic analysis and assistive video overlays. A computer-based video enhancement and video archiving management system providing real-time reference materials, context-sensitive checklists, assistive algorithms, and support for pre- and post-operative reporting. An audio processing system in medical environments for recording vocal bookmarks during a surgical procedure to specifically reference exact moments during a procedure.

### BACKGROUND OF THE INVENTION

Video cameras are a common part of the modern operating room. Some are attached to overhead lights to provide the surgical team with a birds-eye perspective. Others are embedded in devices that surgeons use to view inside a patient's body through small incisions or orifices during minimally invasive surgery. The video recordings from within operating rooms are sometimes used for enhancing medical education, facilitating research activities, promoting communication between healthcare professionals, and advancing patient care. It is not uncommon for a surgeon to study recordings of procedures they have done to improve their skills or refine techniques to improve surgical outcomes. Moreover, real-time video has evolved to play a more active role in guiding and assisting throughout the surgical process through reference materials, checklists, video analytics, computer graphics, and now artificial intelligence to increase precision, safety and efficiency.

Video recordings from the operating room could help solve the frustrating and far-reaching problem of incorrect CPT codes. Doctors and health care professionals use the Current Procedural Terminology, aka CPT® codes as a uniform language for coding medical services and procedures to streamline reporting, increase accuracy and efficiency.

CPT codes are also used for administrative management purposes such as claims processing and developing guidelines for medical care review. However, clerical errors are common, and an expensive problem is created when insurers reject reimbursement claims because of incorrect CPT codes. A rejected claim is sent back to the provider for further documentation and/or justification and resubmission. Depending on the service, rejects are in order of 10% to 25% of submitted claims. Today, sources say there are approximately 3 billion medical insurance claims filed each year. This means there are between 300 and 750 million rejected claims each year. Rejects are a major problem in the insurance and Medicare system that creates an enormous burden on clerical and professional staff, plus slowing down the cash flow to hospitals and medical providers.

The present system is a computer-based video enhancement and archiving management system. In the present invention video plays an active role in the surgical process by providing reference materials, context sensitive checklists, assistive algorithms in real time, and support for pre- and post-operative reporting. The innovations described herein encourage better outcomes through increased safety and efficiency using a video system in an operating room environment that integrates current and emerging medical technology.

### SUMMARY OF THE INVENTION

The present system pertains to a computer-based video enhancement and video archiving management system. The operating room or surgical environment encompass multiple cameras, a room microphone, a computer composed of cameras, microphones, and sensors, connected to a hospital database, a local archive, patient and staff monitors, and various medical sensors such as tactile, blood pressure, heart, acoustic, flow and oximeter sensors. A computer accesses an online database that describes each camera's native format, preprocessing algorithms and the formatting parameters needed to position the image in the final frame to be encoded.

Also available online are context sensitive sequences of Reference Objects (RO's) that automatically appear or disappear as overlays on the video when and while relevant to the then current surgical phase. Reference objects can be real images from medical image scanners, synthetic anatomical drawings, checklists sequenced by surgical phase, step by step instructions for a specific procedure, or any annotative object that should assist with a procedure. Reference objects can be overlaid or chroma-keyed at programmable display coordinates.

The present invention has a video encoding and an audio processing process. Audio stream is captured with video of a surgical procedure. There is a plurality of uses for capturing an audio stream in a surgical environment. One is to enable the surgeon to set verbal bookmarks during the procedure to replay selected sequences. Verbal bookmarks can be said aloud to set bookmarks in a video, such as, “bleeder” to point when a vessel begins squirting blood, “before” when designating a condition before resection, “embed” when identifying a point where an object is embedded in a patient, such as a clip, kirlix, needle, tube, or any surgically assistive object, “timeout” to identify a point where a procedure is paused to assess progress or checkpoint prescribed for the procedure, “follow up” for following up any necessary recommendations, or “report short description” to indicate that the current phase of the procedure should be included in the provided in the database. The system can be customized so that any desired word can be used a bookmark, and in practice, the system creates a video bookmark from a library of keywords, or any words recognized as a noun or other selected part of speech. Adjectives, verbs, adverbs, and prepositions are also captured to support advanced AI analysis planned for future applications.

Another benefit of bookmarking is to create an audit trail that supports medical insurance claims. In certain embodiments, advanced AI analysis can be applied to the documented audit trail and related artifacts to automatically generate the correct CPT codes for a procedure. The healthcare provider uses the CPT code on invoices submitted to insurers for reimbursement. The code describes the service, and the insurer uses it to lookup the then current reimbursement amount to be paid for that specific medical service. Insurers have teams of analysts that examine claims for accuracy and justifiability.

The present system mitigates issues of claim reimbursement by generating a video playback link to support the CPT code used to claim reimbursement. The insurer will receive line-item invoices that include a link to the video saved in the cloud. A claim examiner can then click on the link to replay from the point in the surgical procedure where the CPT coded service is demonstrated. The replay includes the visual and audio that enables the examiner to see the service being performed and hear comments and discussions from the surgical team. The present invention could substantially reduce the number of medical claim rejections and potentially eliminate the unproductive cycle of reject-justify-resubmit that frustrates patients and providers.

Another method of bookmarking would be to use image analysis to detect anomalies in the image characteristics of an anatomic structure. This could be due to variances in color, texture, position, orientation, relative size, rhythmic movement, or other characteristics associated with the structure in view. Computer algorithms would detect the variances by comparing the live view to a synthetic view or descriptive data in an online reference database. A bookmark would be automatically generated should a vessel or organ (artery, vein, ureter, kidney, etc.) exhibit a characteristic outside the parameters provided in the database. The following is an example of an autogenerated bookmark: “Anomaly: possible renal stone detected at post-op written report ureteropelvic junction (UPJ)”. At any point in the procedure, the surgeon could request a display of available anomalous bookmarks by saying “display anomalies”, which would display a list of thumbnail images that the surgeon could select for replay.

Other features and aspects of the invention will become apparent from the following detailed description, taken in conjunction with the accompanying drawings, which illustrate, by way of example, the features in accordance with embodiments of the invention. The summary is not intended to limit the scope of the invention, which is defined solely by the claims attached hereto.

### BRIEF DESCRIPTION OF THE DRAWINGS

The various embodiments are illustrated by way of example, and not by way of limitation, in the figures of the accompanying drawings. Having thus described the invention in general terms, reference will now be made to the accompanying drawings, which are not necessarily drawn to scale, and wherein:

FIG. 1 depicts the present invention utilized in an exemplary environment, an operating room.

FIG. 2 is a depiction of the video encoding process.

FIG. 3 is an example of the audio processing feature of the present system.

FIG. 4 is an illustration depicting an exemplary operating environment, including one or more user computers, computing devices, or processing devices, which can be used to operate a client, such as a dedicated application or web browser.

FIG. 5 is another illustration depicting an exemplary operating environment including a computer system with various elements.

### DETAILED DESCRIPTION OF THE PREFERRED EMBODIMENT

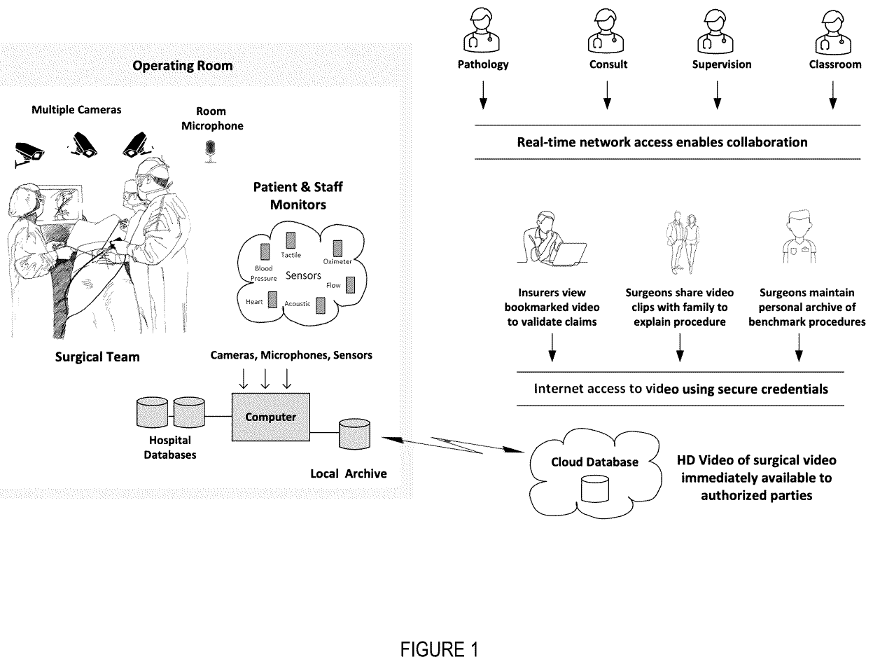

FIG. 1 depicts the present invention utilized in an exemplary environment, an operating room. The system embodies multiple cameras, a room microphone, patient and staff monitors and sensors. Some of the sensors, by way of example and not limitation, include tactile, blood pressure, heart, acoustic, oximeter, flow, acoustic, and all necessary medical sensors that are used for surgical procedures, such as force, optical, gyroscopes, capnography, ultrasound and/or thermal, sensors. A computer with a processing device is used to access hospital databases, save and retrieve videos gathered from the procedure on local and cloud databases, and make them available to authorized parties. The present invention operates through a secure network that allows real-time cross-collaboration across various departments using secure credentials. For example, insurers can view bookmarked commands in the video to validate claims, surgeons can share clips, annotate the anatomical structure of an operation, and explain a procedure using a keyboard connected to the display. Surgeons can also maintain a personal archive of benchmark procedures and use keystroke shortcuts to execute a series of commands, such as generate a text summary of the operation, compare the vide of an anatomic structure, and display reference objects. In addition, with proper approvals procedures can be used for research or shown to medical students/residents for training.

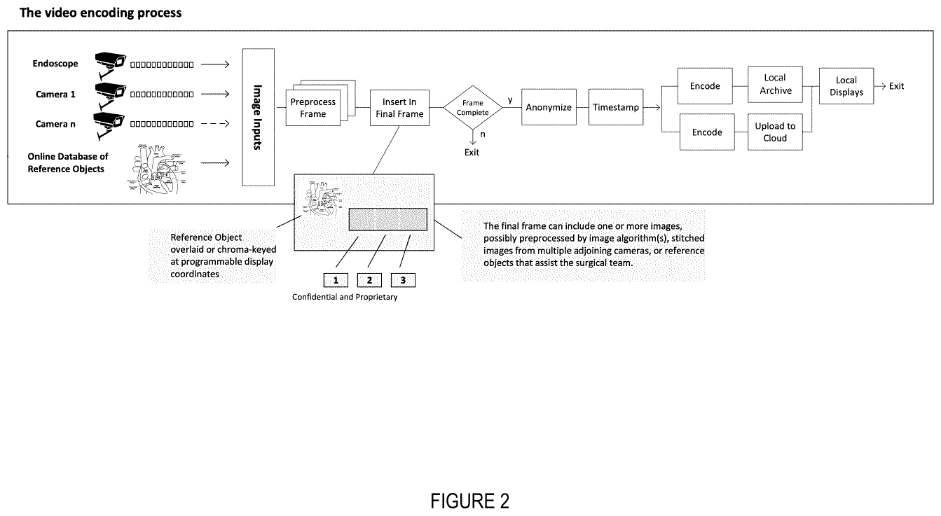

FIG. 2 is a depiction of the video encoding process. One or more cameras can appear in multiple embodiments. For example, a camera may be mounted in the corner of a surgical operating environment, an endoscope, a surgical device camera, or a mounted camera for close view and examination of a patient or surgical workspace. In any case, the image input derives from a camera stream or video stream from a video streaming device, such as a video camera, web camera, or electronic device with a camera. The image inputs include pre-processed frames and reference objects frames may be be overlayed or chroma-keyed at programmable display coordinate. Reference objects can be from previous media, such as a hospital database, local database, or internet database, and can be overlayed on the desired media or frame to assist a surgical team. The image can then be timestamped, anonymized, and encoded then uploaded to a local and cloud archive. Moreover, a user can create a compositive video frame by way of stitching a multitude of input video inputs from cameras corresponding to a video display of a surgical procedure. A user can add latency, control whether the image presented is pre or post processing, and broadcast the video stream with processing algorithms executed in real time.

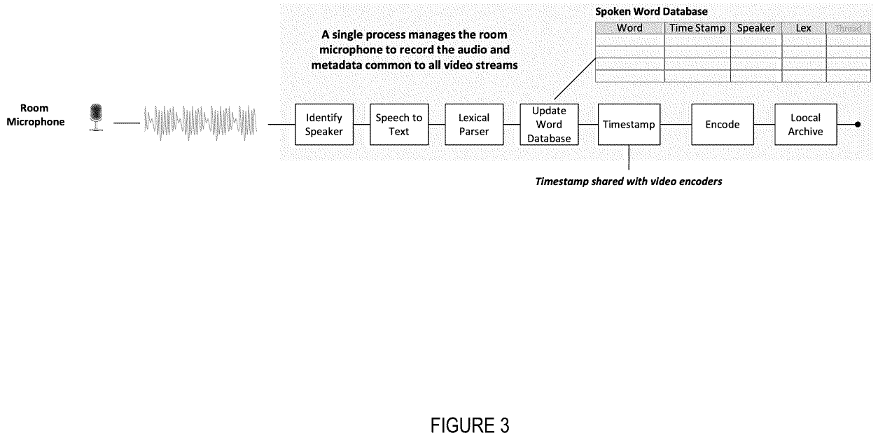

FIG. 3 is an example of the audio processing feature of the present system. A single process manages the room microphone to record the audio and metadata from the video and video streams. The room microphone identifies the speaker and converts the speaker's speech to text. The text undergoes lexical parsing and updates the spoken word database with the timestamp, speaker data, and lexical parser data. All the data is then encoded and stored on a local and cloud system archive and random-access memory. The archived data can be used to train an artificial intelligence algorithm, or referenced by personnel and claims insurers.

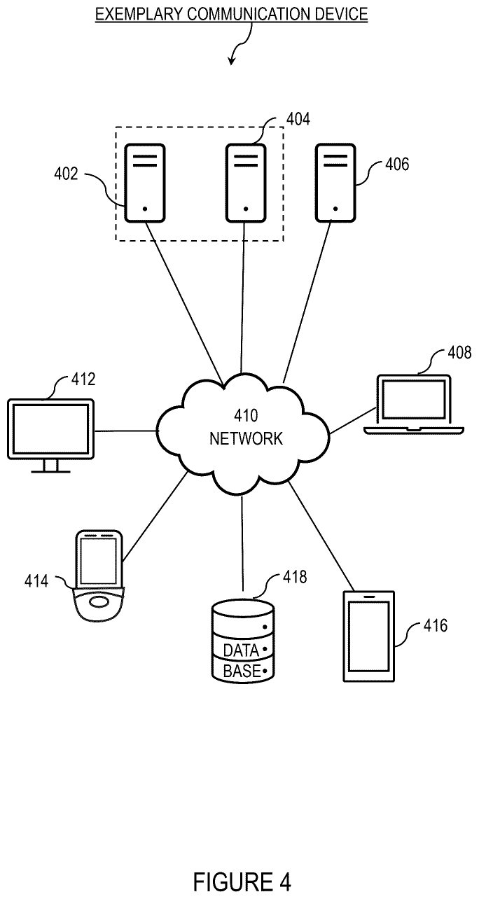

FIG. 4 shows the web services of the platform and system. The platform and system are all components of an exemplary operating environment where embodiments of the present invention may be implemented. The system can include one or more user computers, computing devices, or processing devices that can operate a client, such as a dedicated application, web browser, etc. The user computers 408, 412 can be general purpose personal computers 408, 412 (including, merely by way of example, personal computers and/or laptop computers running a standard operating system), cell phones or tablets 414, 416, (running mobile software and being Internet, e-mail, SMS, or other communication protocol enabled), and/or workstation, computers running any of a variety of commercially-available UNIX or UNIX-like operating systems (including without limitation, the variety of GNU/Linux operating systems). These user computers may also have any of a variety of applications, including one or more development systems, database client and/or server applications, and Web browser applications. Alternatively, the user computers may be any other electronic device, capable of communicating via a network 410 (e.g., the network described below) and/or displaying and navigating Web pages or other types of electronic documents, specifically pertaining to clients and their surgical operations. Although the exemplary system is shown with four user computers, any number of user computers may be supported.

In most embodiments, the system includes some type of network 410. The network 410 can be any type of network 410 familiar to those skilled in the art that can support data communications using any of a variety of commercially-available protocols, including without limitation TCP/IP, SNA, IPX, AppleTalk, and the like. Merely by way of example, the network can be a local area network (“LAN”), such as an Ethernet network, a Token-Ring network and/or the like; a wide-area network; a virtual network, including without limitation a virtual private network (“VPN”); the Internet; an intranet; an extranet; a public switched telephone network (“PSTN”); an infra-red network; a wireless, the Bluetooth protocol known in the art for configuring multiple devices, and/or any other wireless protocol); and/or any combination of these and/or other networks.

The system may also include one or more server computers 402, 404, 406 which can be general purpose computers, specialized server computers (including, merely by way of example, PC servers, UNIX servers, mid-range servers, mainframe computers rack-mounted servers, etc.), server farms, server clusters, or any other appropriate arrangement and/or combination. One or more of the servers may be dedicated to running applications, such as a business application, a Web server, application server, etc. Such servers may be used to process requests from user computers. The applications can also include any number of applications for controlling access to resources of the servers.

FIG. 4 further illustrates an environment where an on-demand distributed 418 service might be used. As illustrated in FIG. 4 user systems might interact via a network with an on-demand database. Some on-demand databases may store information from one or more records stored in tables of one or more distributed database images to form a database management system (DBMS). Accordingly, on-demand database and system will be used interchangeably herein. A database image may include one or more database objects. A relational database management system (RDMS) or the equivalent may execute storage and retrieval of information against the database object(s). Some on-demand database services may include an application platform that enables the creation, managing, and execution of one or more applications developed by the provider of the on-demand database service, wherein users access the on-demand database service via user systems or third-party application developers access the on-demand database service via user systems.

The security of a particular user system might be entirely determined by permissions (permission levels) for the current user. For example, where a user account identification transaction may involve a portable identification alpha-numeric data field physically or digitally linked to a personal primary identification device to request services from a provider account and wherein the user is using a particular user system to interact with System, that user system has the permissions allotted to that user account. However, while an administrator uses that user system to interact with System, that user system has the permissions allotted to that administrator. In systems with a hierarchical role model, users at one permission level may have access to applications, data, and database information accessible by a lower permission level user, but may not have access to certain applications, database information, and data accessible by a user at a higher permission level. Thus, different users will have different permissions with regard to accessing and modifying application and database information, depending on a user's security or permission level.

A network can be a LAN (local area network), WAN (wide area network), wireless network, point-to-point network, star network, token ring network, hub network, or another appropriate configuration. As the most common type of network in current use is a TCP/IP (Transfer Control Protocol and Internet Protocol) network such as the global internetwork of networks often referred to as the “Internet” with a capital “I,” that will be used in many of the examples herein. However, it should be understood that the networks that the present invention might use are not so limited, although TCP/IP is a frequently implemented protocol.

User systems might communicate with a system using TCP/IP and, at a higher network level, use other common Internet protocols to communicate, such as HTTP, FTP, AFS, WAP, etc. In an example where HTTP is used, a user system might include an HTTP client commonly referred to as a “browser” for sending and receiving HTTP messages to and from an HTTP server at System. Such HTTP server might be implemented as the sole network interface between a system and network, but other techniques might be used as well or instead. In some implementations, the interface between a system and network includes load sharing functionality, such as round-robin HTTP request distributors to balance loads and distribute incoming HTTP requests evenly over a plurality of servers. At least as for the users that are accessing that server, each of the plurality of servers has access to at least one third-party entity system data schema; however, other alternative configurations are contemplated.

According to one arrangement, each user system and all its components are operator configurable using applications, such as a browser, including computer code run using a central processing unit or processor. Similarly, a computer system (and additional instances of an enterprise database, where more than one is present) and all their components might be operator configurable using application(s) including computer code run using a central processing unit or processor, or multiple processor units. A computer program product aspect includes a machine-readable storage medium (media) having instructions stored thereon/in which can be used to program a computer to perform any of the processes of the embodiments described herein. Computer code for operating and configuring systems to intercommunicate and to process web pages, applications and other data and media content as described herein is preferably downloaded and stored on a hard disk, but the entire program code, or portions thereof, may also be locally stored in any other volatile or non-volatile memory medium or device as is well known, such as a ROM or RAM, or provided on any media capable of storing program code, or any type of media or device suitable for storing instructions and/or data. Additionally, the entire program code, or portions thereof, may be transmitted and downloaded from a software source over a transmission medium, e.g., over the Internet, or from another server, as is well known, or transmitted over any other conventional network connection as is well known (e.g., extranet, VPN, LAN, etc.) using any communication medium and protocols (e.g., TCP/IP, HTTP, HTTPS, Ethernet, etc.) as are well known. It will also be appreciated that computer code for implementing aspects of the present invention can be implemented in any programming language that can be executed on a client system and/or server or server systems.

The Web server can be running an operating system 400 including any of those discussed above, as well as any commercially-available server operating systems. The Web server can also run any of a variety of server applications and/or mid-tier applications, including HTTP servers, FTP servers, CGI servers, database servers, Java servers, business applications, and the like. The server(s) also may be one or more computers which can be capable of executing programs or scripts in response to the user computers. As one example, a server may execute one or more Web applications. The Web application may be implemented as one or more scripts or programs written in any programming language. The server(s) may also include database servers, including without limitation those commercially available, which can process requests from database clients running on a user computer.

End users, or users that are viewing and using the network platform, all contribute data to the cloud. A web service platform helps secure that data and maintain the service's functionalities. Only authorized users and entities can authorize or unauthorize content and monitor data stored within the web service. The platform's web services help maintain the operations of elements managed by the storage system.

The system may also include one or more databases 420. The database(s) may reside in a variety of locations. By way of example, a database 418 may reside on a storage medium local to (and/or resident in) one or more of the computers. Alternatively, it may be remote from any or all of the computers, and/or in communication (e.g., via the network) with one or more of these. In a particular set of embodiments, the database may reside in a storage-area network (“SAN”) familiar to those skilled in the art. Similarly, any necessary files for performing the functions attributed to the computers may be stored locally on the respective computer and/or remotely, as appropriate. In one set of embodiments, the database may be a relational database, such as Oracle 10g, that is adapted to store, update, and retrieve data in response to SQL-formatted commands.

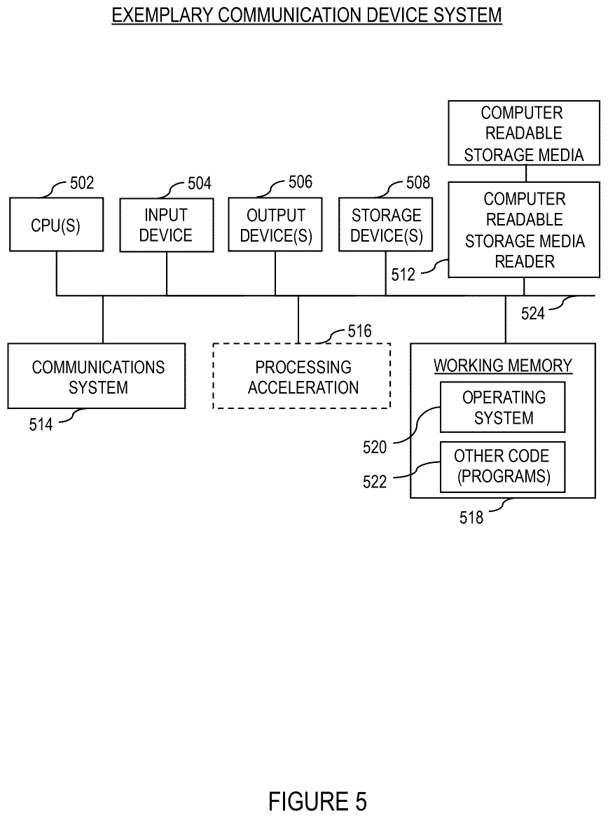

FIG. 5 illustrates an exemplary computer system, in which embodiments of the present invention may be implemented. The system 500 may be used to implement any of the computer systems described above. The computer system 500 is shown comprising hardware elements that may be electrically coupled via a bus. The hardware elements may include one or more central processing units 502 (CPUs) configured to a graphic display, one or more input devices 504 (e.g., a mouse, a keyboard, etc.), and one or more output devices 506 (e.g., a display device, a printer, etc.). The computer system 500 may also include one or more storage devices 508. By way of example, the storage device(s) can include devices such as disk drives, optical storage devices, solid-state storage device such as a random-access memory (“RAM”) and/or a read-only memory (“ROM”), which can be programmable, flash-updateable and/or the like, as well as non-transitory and transitory storage mediums.

The computer system 500 may additionally include a computer-readable storage media reader 512, a communications system 514 (e.g., a modem, a network card (wireless or wired), an infra-red communication device, etc.), and working memory 518, which may include RAM and ROM devices as described above. In some embodiments, the computer system may also include a processing acceleration unit 516, which can include a digital signal processor DSP, a special-purpose processor, and/or the like.

The computer-readable storage media reader 512 can further be connected to a computer-readable storage medium, together (and, optionally, in combination with storage device(s)) comprehensively representing remote, local, cloud, fixed, and/or removable storage devices plus storage media for temporarily and/or more permanently containing, storing, transmitting, and retrieving computer-readable information. The communications system may permit data to be exchanged with the network and/or any other computer described above with respect to the system and valid credentials offered by a secure third party.

The computer system may also comprise software elements, shown as being currently located within a working memory, including an operating system 520 and/or other code 522, such as an application program (which may be a client application, Web browser, mid-tier application, RDBMS, etc.). It should be appreciated that alternate embodiments of a computer system may have numerous variations from that described above. For example, customized hardware might also be used and/or particular elements might be implemented in hardware, software (including portable software, such as applets), or both. Further, connection to other computing devices such as network input/output devices may be employed.

Storage media and computer readable media 510 for containing code 522, or portions of code, can include any appropriate media known or used in the art, including storage media and communication media, such as but not limited to volatile and non-volatile, removable and non-removable media implemented in any method or technology for storage and/or transmission of information such as computer readable instructions, data structures, program modules, or other data, including RAM, ROM, EEPROM, flash memory or other memory technology, CD-ROM, digital versatile disk (DVD) or other optical storage, such as a cloud database, disk storage or other magnetic storage devices, data signals, data transmissions, or any other medium which can be used to store or transmit the desired information and which can be accessed by the computer. Based on the disclosure and teachings provided herein, a person of ordinary skill in the art will appreciate other ways and/or methods to implement the various embodiments.

As discussed above, embodiments are suitable for use with the Internet, which refers to a specific global internetwork of networks. However, it should be understood that other networks can be used instead of the Internet, such as an intranet, an extranet, a virtual private network (VPN), a non-TCP/IP based network, any LAN or WAN or the like. For example, an anonymized user can access the interface through a secure virtual private network in order to ensure patient confidentiality.

While various embodiments of the disclosed technology have been described above, it should be understood that they have been presented by way of example only, and not of limitation. Likewise, the various diagrams may depict an example architectural or other configuration for the disclosed technology, which is done to aid in understanding the features and functionality that may be included in the disclosed technology. The disclosed technology is not restricted to the illustrated example architectures or configurations, but the desired features may be implemented using a variety of alternative architectures and configurations. Indeed, it will be apparent to one of skill in the art how alternative functional, logical or physical partitioning and configurations may be implemented to implement the desired features of the technology disclosed herein. Also, a multitude of different constituent module names other than those depicted herein may be applied to the various partitions. Additionally, with regard to flow diagrams, operational descriptions and method claims, the order in which the steps are presented herein shall not mandate that various embodiments be implemented to perform the recited functionality in the same order unless the context dictates otherwise.

Although the disclosed technology is described above in terms of various exemplary embodiments and implementations, it should be understood that the various features, aspects and functionality described in one or more of the individual embodiments are not limited in their applicability to the particular embodiment with which they are described, but instead may be applied, alone or in various combinations, to one or more of the other embodiments of the disclosed technology, whether or not such embodiments are described and whether or not such features are presented as being a part of a described embodiment. Thus, the breadth and scope of the technology disclosed herein should not be limited by any of the above-described exemplary embodiments.

Terms and phrases used in this document, and variations thereof, unless otherwise expressly stated, should be construed as open ended as opposed to limiting. As examples of the foregoing: the term “including” should be read as meaning “including, without limitation” or the like; the term “example” is used to provide exemplary instances of the item in discussion, not an exhaustive or limiting list thereof; the terms “a” or “an” should be read as meaning “at least one,” “one or more” or the like; and adjectives such as “conventional,” “traditional,” “normal,” “standard,” “known” and terms of similar meaning should not be construed as limiting the item described to a given time period or to an item available as of a given time, but instead should be read to encompass conventional, traditional, normal, or standard technologies that may be available or known now or at any time in the future. Likewise, where this document refers to technologies that would be apparent or known to one of ordinary skill in the art, such technologies encompass those apparent or known to the skilled artisan now or at any time in the future.

What is claimed is:

1. A system for monitoring and recording a surgical environment with a real-time algorithmic analysis and video overlays, comprising:
a plurality of cameras configured to capture visual data in a medical environment, and, wherein at least one of said plurality of cameras feature a microphone to capture audio data from within said medical environment;
a plurality of sensors, configured to a medical device to receive and monitor patient data; and
a computer comprising a processing unit and memory unit, configured to receive said patient data from said plurality of sensors, said visual data and said audio data from said plurality of cameras, and wherein said processing unit is instructed to execute a video encoding process to:
receive an image input from said plurality of cameras, and a reference object from an online data base;
overlay said reference object in proximity to said image input at a programmable display's coordinates;
receive an audio input from at least one of said plurality of cameras with said microphone;
convert said audio input to an audio output with lexically-parsed text;
transmit said lexically-parsed text to a spoken word database; and
train an artificial intelligence algorithm using said spoken word database.

2. The system of claim 1, wherein at least one of said plurality of sensors is a pressure, heart, acoustic, oximeter, flow, optical, gyroscope, capnography, ultrasound and thermal sensor.

3. The system of claim 1, wherein said computer operates over a secure network to receive and transmit said visual data, said audio data and said patient data.

4. The system of claim 1, wherein at least one of said plurality of cameras is mounted on a surgical device for close view and examination of a patient and surgical workspace.

5. The system of claim 1, further comprising of said reference object chroma-keyed at said programmable display's coordinates.

6. A method for monitoring and recording a surgical procedure with a real-time algorithmic analysis and video overlays comprising:
capturing visual data in a medical environment using a plurality of cameras, and wherein at least one of said plurality of cameras feature a microphone to capture audio data from within said medical environment;
receiving patient data deriving from a plurality of sensors, configured to a medical device; and
configuring a computer comprising a processing unit and memory unit to receive said patient data from said plurality of sensors, said visual data and said audio data from said plurality of cameras, and wherein said processing unit is instructed to execute video encoding process for:
receiving an image input from said plurality of cameras, and a reference object from an online database;
overlaying said reference object in proximity to said image input at a programmable display's coordinates;
receiving an audio input from at least one of said plurality of cameras with said microphone;
converting said audio input to an audio output with lexically-parsed text;
transmitting said lexically-parsed text to a spoken word database; and
training an artificial intelligence algorithm using said spoken word database.

7. The method of claim 6, wherein at least one of said plurality of sensors is a pressure, heart, acoustic, oximeter, flow, optical, gyroscope, capnography, ultrasound and thermal sensor.

8. The method of claim 6, wherein said computer operates over a secure network to receive and transmit said visual data, said audio data and said patient data.

9. The method of claim 6, wherein at least one of said plurality of cameras is mounted on a surgical device for close view and examination of a patient and surgical workspace.

10. The method of claim 6, further comprising of said reference object chroma-keyed on said programmable display's coordinates.

11. The method of claim 6, wherein said reference object are images from medical image scanners, synthetic anatomical drawings, checklists sequenced by surgical phase, step by step instructions for a specific procedure, or an annotative object that assists with a procedure.

12. The method of claim 6, wherein said microphone can captures said audio data to execute an action through said processing unit configured to said computer in response to a verbal bookmark.

13. The method of claim 12, wherein said audio data is used to create an audio trail to support a medical insurance claim.

14. The method of claim 13, wherein said artificial intelligence algorithm assesses said audio trail to generate a CPT code for said surgical procedure.

15. A computer-implemented system for monitoring and recording a procedure with real-time algorithmic analysis and video overlays, comprising:
a plurality of cameras configured to capture visual data in a medical environment;
wherein at least one of said plurality of cameras feature a microphone to capture audio data from within said medical environment, and at least one of said plurality of cameras are mounted on a surgical device for close view and examination of a patient and surgical workspace;
a plurality of sensors, configured to a medical device to receive and monitor patient data; and
a computer comprising a processing unit and memory unit, configured to receive said patient data from said plurality of sensors, said visual data and said audio data from said plurality of cameras over a secure network, and wherein said processing unit is instructed to execute a video encoding process to:
receive an image input from said plurality of cameras;
select an overlay from a plurality of reference objects from an online database, and wherein at least one of said plurality of reference objects derive from an image scan, synthetic anatomical drawing, a surgical checklist sequenced by surgical phase, an instruction guide for a surgical procedure, and an annotative object to assist with said surgical procedure;
overlay at least one of said plurality of reference objects in proximity to said image input at coordinates of a programmable display to generate an image output;
receive an audio input from at least one of said plurality of cameras with said microphone;
convert said audio input to an audio output with lexically-parsed text;
transmit said lexically-parsed text to a spoken word database; and
train an artificial intelligence algorithm using said spoken word database to execute an action to modify said image output using a verbal bookmark.

16. The computer-implemented system of claim 15, wherein at least one of said plurality of sensors is a pressure, heart, acoustic, oximeter, flow, optical, gyroscope, capnography, ultrasound and thermal sensor.

17. The computer-implemented system of claim 15, wherein said spoken word database enables customization so a user may appoint a designated word as a bookmark using a library of keywords that modify a viewing mode on said programmable display.

18. The computer-implemented system of claim 15, wherein a live view and synthetic view is generated and processed by said computer to compare differences in characteristics independent of anatomical parameters stored in said online database to detect anomalous activity in an examined anatomical structure.

19. The computer-implemented system of claim 15, wherein a local archive and cloud archive is utilized by said secure network for data transmission.

20. The computer-implemented system of claim 15, wherein said image output is timestamped and anonymized.
